(19) United States
(12) Patent Application Publication
(10) Pub. No.: US 2025/0196021 A1
(43) Pub. Date: Jun. 19, 2025

(54) METHODS OF SOLVENT DISPLACEMENT FOR METAL-CONTAINING IONIC LIQUIDS

(71) Applicant: Chevron Phillips Chemical Company LP, The Woodlands, TX (US)

(72) Inventor: Orson L. Sydora, Kingwood, TX (US)

(21) Appl. No.: 18/978,085

(22) Filed: Dec. 12, 2024

Related U.S. Application Data
(60) Provisional application No. 63/609,660, filed on Dec. 13, 2023.

(51) Int. Cl. B01D 3/34; B01D 53/14

(52) U.S. Cl. CPC B01D 3/346; B01D 53/1493; B01D 2252/30; B01D 2256/24

(57) ABSTRACT

Purification processes include a step of contacting a metal-containing ionic liquid composition with a gaseous unsaturated compound at a pressure of at least 50 psig to remove at least a portion of the solvent to form a purified metal-containing ionic liquid composition. The metal-containing ionic liquid composition contains an ionic liquid comprising an ionic liquid cation and an ionic liquid anion, a metal cation, and a solvent at a molar ratio of solvent: metal cation of at least 0.25:1.

### REFERENCE TO RELATED APPLICATION

This application claims the benefit of U.S. Provisional Patent Application No. 63/609,660, filed on Dec. 13, 2023, the disclosure of which is incorporated herein by reference in its entirety.

### FIELD OF THE DISCLOSURE

The present disclosure generally relates to processes for purifying metal-containing ionic liquid compositions, and more particularly relates to displacing solvents that are present in the metal-containing ionic liquid compositions.

### BACKGROUND

Metal-containing ionic liquids have found utility for the capture and recovery of certain compounds from process streams. For instance, systems have been developed to contact metal-containing ionic liquids with process streams to capture ethylene through x-bonding interactions with the metal-containing ionic liquids. However, the metal-containing ionic liquids often have high viscosities that require significant amounts of a solvent or viscosity modifier in order to improve handling, transport, and flowability of the metal-containing ionic liquid.

While the addition of solvents or other viscosity modifiers advantageously results in a less viscous composition, purity of the metal-containing ionic liquid and any product prepared therefrom are negatively impacted. It would be beneficial to develop techniques for processing these metal-containing ionic liquids that overcome these drawbacks. According, it is to these ends that the present disclosure is generally directed.

### SUMMARY

This summary is provided to introduce a selection of concepts in a simplified form that are further described below in the detailed description. This summary is not intended to identify required or essential features of the claimed subject matter. Nor is this summary intended to be used to limit the scope of the claimed subject matter.

Aspects of this disclosure are directed to processes that comprise a step of contacting a metal-containing ionic liquid composition-which can contain (i) an ionic liquid comprising an ionic liquid cation and an ionic liquid anion, (ii) a metal cation, and (iii) a solvent at a molar ratio of solvent: metal cation of at least 0.25:1—with a gaseous unsaturated compound at a pressure of at least 50 psig to remove at least a portion of the solvent to form a purified metal-containing ionic liquid composition.

Beneficially, the metal-containing ionic liquid composition can contain a suitable amount of solvent for ease of handling, transport, and flowability. Then, prior to use, the solvent can conveniently be displaced, thereby increasing the purity of the metal-containing ionic liquid composition and thus any products that are derived from utilizing the metal-containing ionic liquid composition. A particular use of the metal-containing ionic liquid composition described herein is to selectively remove and recover ethylene versus ethane from process streams.

Both the foregoing summary and the following detailed description provide examples and are explanatory only. Accordingly, the foregoing summary and the following detailed description should not be considered to be restrictive. Further, features or variations may be provided in addition to those set forth herein. For example, certain aspects may be directed to various feature combinations and sub-combinations described in the detailed description.

### DEFINITIONS

To define more clearly the terms used herein, the following definitions are provided. Unless otherwise indicated, the following definitions are applicable to this disclosure. If a term is used in this disclosure but is not specifically defined herein, the definition from the IUPAC Compendium of Chemical Terminology, 2^{nd} Ed (1997), can be applied, as long as that definition does not conflict with any other disclosure or definition applied herein, or render indefinite or non-enabled any claim to which that definition is applied. To the extent that any definition or usage provided by any document incorporated herein by reference conflicts with the definition or usage provided herein, the definition or usage provided herein controls.

Herein, features of the subject matter are described such that, within particular aspects, a combination of different features can be envisioned. For each and every aspect and each and every feature disclosed herein, all combinations that do not detrimentally affect the processes or methods described herein are contemplated with or without explicit description of the particular combination. Additionally, unless explicitly recited otherwise, any aspect or feature disclosed herein can be combined to describe inventive compositions or methods consistent with the present disclosure.

For any particular compound or group disclosed herein, any name or structure (general or specific) presented is intended to encompass all conformational isomers, regioisomers, stereoisomers, and mixtures thereof that can arise from a particular set of substituents, unless otherwise specified. The name or structure (general or specific) also encompasses all enantiomers, diastereomers, and other optical isomers (if there are any) whether in enantiomeric or racemic forms, as well as mixtures of stereoisomers, as would be recognized by a skilled artisan, unless otherwise specified. For instance, a general reference to pentane includes n-pentane, 2-methyl-butane, and 2,2-dimethylpropane; and a general reference to a butyl group includes a n-butyl group, a sec-butyl group, an iso-butyl group, and a t-butyl group.

The terms “contacting” and “combining” are used herein to describe compositions and methods in which the materials or components are contacted or combined together in any order, in any manner, and for any length of time, unless otherwise specified. For example, the materials or components can be blended, mixed, slurried, dissolved, reacted, treated, impregnated, compounded, or otherwise contacted or combined in some other manner or by any suitable method or technique.

In this disclosure, while compositions and methods are described in terms of “comprising” various components or steps, the compositions and methods also can “consist essentially of” or “consist of” the various components or steps, unless stated otherwise. The terms “a,” “an,” and “the” are intended to include plural alternatives, e.g., at least one, unless otherwise specified.

Several types of ranges are disclosed in the present disclosure. When a range of any type is disclosed or claimed, the intent is to disclose or claim individually each possible number that such a range could reasonably encompass, including end points of the range as well as any sub-ranges and combinations of sub-ranges encompassed therein. For example, metal-containing ionic liquid compositions disclosed herein can be characterized by a molar ratio of solvent: metal cation of at least 0.25:1. By a disclosure that the solvent: metal cation molar ratio can be at least 0.25:1, the intent is to recite that the molar ratio can be any amount that is at least 0.25:1 and, for example, can include any range or combination of ranges that are least 0.25:1, such as from 0.25:1 to 5:1, from 0.25:1 to 3:1, from 0.25:1 to 1:1, from 0.5:1 to 5:1, from 0.5:1 to 3:1, from 0.5:1 to 1:1, or from 1:1 to 2:1, and so forth. Likewise, all other ranges disclosed herein should be interpreted in a manner similar to this example.

In general, an amount, size, formulation, parameter, range, or other quantity or characteristic is “about” or “approximate” whether or not expressly stated to be such. Whether or not modified by the term “about” or “approximately,” the claims include equivalents to the quantities or characteristics.

The term “ionic liquid” (or “IL”) as used herein is given its ordinary meaning in the art, and means liquids, more particularly molten salts, that contain substantially only ions, and more particularly only anions and cations, having a melting point of 200° C. or less, and having a high thermal stability and a low flammability. Room Temperature Ionic Liquids (RTILs) are ionic liquids that are liquid at standard temperature and pressure, preferably below 100° C. In certain embodiments, the ionic liquid may be represented by the empirical formula AₓQy, where A is a monoanion or polyanion, Q is a monocation or polycation, x is a number ranging from 1 to about 4, and y is a number ranging from 1 to about 4. The cations associated with ILs are structurally diverse, but generally contain one or more nitrogens as part of a ring structure and are capable of being converted to quaternary amines. Examples of such cations include pyridinium, pyridazinium, pyrimidinium, pyrazinium, imidazolium, pyrazolium, oxazolium, triazolium, thiazolium, piperidinium, pyrrolidinium, quinolinium, and isoquinolinium. Anions bound to ILs can also be structurally diverse and varied and can have a significant impact on the solubility of ILs in different media. For example, ILs containing hydrophobic anions such as hexafluorophosphate or trifluoromethanesulfonimides have very low solubility in water, whereas ILs containing hydrophilic anions such as chloride or acetate are completely miscible in water. The name of ionic liquids may generally be abbreviated. Alkyl cations are usually named by the alkyl substituent and the letter of the cation, which are given in a set of brackets, followed by the abbreviation of the anion. Although not expressly written, it is understood that the cation has a positive charge and the anion has a negative charge. For example, [BMIm] OAc represents 1-butyl-3-methylimidazolium acetate, [AMIm] Cl represents 1-allyl-3-methylimidazolium chloride, and [EMIm] OF represents 1-ethyl-3-methylimidazolium formate.

The term metal-containing ionic liquid “composition” is used herein to describe a composition comprising an ionic liquid as a majority component, and therefore can be applied to compositions generally that contain more than 50 wt. % ion pairs and exist as a liquid under standard temperature and pressure. Metal-containing ionic liquid compositions can therefore be understood to refer generally to compositions including, but not limited to, an ionic liquid, a metal cation, and a solvent.

Although any methods, devices, and materials similar or equivalent to those described herein can be used in the practice or testing of the disclosure, the typical methods, devices, and materials are herein described.

All publications and patents mentioned herein are incorporated herein by reference in their entirety for the purpose of describing and disclosing, for example, the constructs and methodologies that are described in the publications and patents, which might be used in connection with the presently described disclosure.

### DETAILED DESCRIPTION

The high viscosity of a metal-containing ionic liquid composition leads to difficulties with handling, transport, and flowability of the composition. Therefore, the metal-containing ionic liquid composition contains a solvent (e.g., a coordinating solvent) to reduce the viscosity of the composition and to improve handling, transport, and flowability of the composition.

Solvent addition, however, negatively impacts the purity of the metal-containing ionic liquid and any product prepared therefrom. If not removed prior to the use of the metal-containing ionic liquid composition, the solvent can end up in the final product stream, which is undesirable.

One method for removing solvent is by thermal treatment, such as by heating to over 140° C. and evaporation of the solvent. Another method to remove solvent is by exposing the composition to reduced pressure (e.g., applying a vacuum). Yet another method is by utilizing a combination of thermal treatment with lower pressure. These methods are very energy intensive and often can take days to remove the tightly bound solvent to acceptable levels.

An objective of this disclosure, therefore, is to develop an alternative technique for removing solvent from, and for the purification of, a metal-containing ionic liquid composition. Briefly, coordinating solvent present in a metal-containing ionic liquid composition is removed by displacing the solvent with a gaseous unsaturated compound, thereby removing solvent and forming a purified metal-containing ionic liquid composition. Subsequently, the removed solvent and excess gaseous unsaturated compound are vented. Multiple cycles may be used to reduce the solvent content of the metal-containing ionic liquid composition to any desirable level.

### Metal-Containing Ionic Liquid Compositions

The processes disclosed herein generally are utilized to purify metal-containing ionic liquid compositions. These compositions can contain (i) an ionic liquid comprising an ionic liquid cation and an ionic liquid anion, (ii) a metal cation, and (iii) a solvent. Typically, the ionic liquid constitutes the majority of the metal-containing ionic liquid composition with the composition containing at least 50 wt. % of the ionic liquid, and more often, at least 60 wt. %, at least 75 wt. %, at least 90 wt. %, or at least 95 wt. % of the ionic liquid.

The ionic liquid comprises an ionic liquid cation and an ionic liquid anion. While not limited thereto, the ionic liquid cation can comprise, for instance, an allylmethylimidazolium, a butylethylimidazolium, a butylmethylimidazolium, a butyldimethylimidazolium, a decaethylimidazolium, a decamethylimidazolium, a diethylimidazolium, a dimethylimidazolium, an ethyl-2,4-dimethylimidazolium, an ethyldimethylimidazolium, an ethylimidazolium, an ethylmethylimidazolium, an ethylpropylimidazolium, an ethoxyethylmethylimidazolium, an ethoxydimethylimidazolium, a hexadecylmethylimidazolium, a heptylmethylimidazolium, a hexylethylimidazolium, a hexylmethylimidazolium, a hexyldimethylimidazolium, a methoxyethylmethylimidazolium, a methoxypropylmethylimidazolium, a methylimidazolium, a dimethylimidazolium, a methylnonylimidazolium, a methylnonylimidazolium, an octadecylmethylimidazolium, a hydroxylethylmethylimidazolium, a hydroxyloctylmethylimidazolium, a hydroxylpropylmethylimidazolium, an octylmethylimidazolium, an octyldimethylimidazolium, a phenylethylmethylimidazolium, a phenylmethylimidazolium, a phenyldimethylimidazolium, a pentylmethylimidazolium, a propylmethylimidazolium, 1-butyl-2-methylpyridinium, 1-butyl-3-methylpyridinium, a butylmethylpyridinium, 1-butyl-4-dimethylacetylpyridinium, 1-butyl-4-methylpyridinium, 1-ethyl-2-methylpyridinium, 1-ethyl-3-methylpyridinium, 1-ethyl-4-dimethylacetylpyridinium, 1-ethyl-4-methylpyridinium, 1-hexyl-4-dimethylacetylpyridinium, 1-hexyl-4-methylpyridinium, 1-octyl-3-methylpyridinium, 1-octyl-4-methylpyridinium, 1-propyl-3-methylpyridinium, 1-propyl-4-methylpyridinium, a butylpyridinium, an ethylpyridinium, a heptylpyridinium, a hexylpyridinium, a hydroxypropylpyridinium, an octylpyridinium, a pentylpyridinium, a propylpyridinium, a butylmethylpyrrolidinium, a butylpyrrolidinium, a hexylmethylpyrrolidinium, a hexylpyrrolidinium, an octylmethylpyrrolidinium, an octylpyrrolidinium, a propylmethylpyrrolidinium, a butylammonium, tributylammonium, tetrabutylammonium, butylethyldimethylammonium, butyltrimethylammonium, N,N,N-trimethylethanolammonium, ethylammonium, diethylammonium, tetraethylammonium, tetraheptylammonium, tetrahexylammonium, methylammonium, dimethylammonium, tetramethylammonium, ammonium, butyldimethylethanolammonium, dimethylethanolammonium, ethanolammonium, ethyldimethylethanolammonium, a tetrapentylammonium, tetrapropylammonium, a tetrabutylphosphonium, a tributyloctylphosphonium, and the like. Combinations of two or more of these ionic liquid cations can be present in the metal-containing ionic liquid composition. Representative and non-limiting examples of suitable ionic liquid

anions can include bis(trifluoromethanesulfonyl)imide, bis(fluorosulfonyl)imide, hexafluorophosphate, trifluoromethanesulfonate, dicyanamide, tetrafluoroborate, thiocyanate, nitrate, sulfonate, methylsulfate, methanesulfonate, trifluoroacetate, acetate, and the like. As with the ionic liquid cation, combinations of two or more of the ionic liquid anions can be present in the metal-containing ionic liquid composition. Typically, the molar ratio of the ionic liquid cation to the ionic liquid anion is approximately 1:1.

In an aspect, the ionic liquid cation can comprise an allylmethylimidazolium, an ethylmethylimidazolium, (1-(2-cyanoethyl))-3-methylimidazolium, a propenylmethylimidazolium, a propargylmethylimidazolium, a butylmethylimidazolium, a butylmethylpyridinium, and the like, as well as any combination thereof. Additionally or alternatively, the ionic liquid anion can comprise bis(trifluoromethanesulfonyl)imide, hexafluorophosphate, trifluoromethanesulfonate, dicyanamide, tetrafluoroborate, thiocyanate, nitrate, sulfonate, methylsulfate, and the like, as well as any combination thereof.

In another aspect, the ionic liquid cation can comprise (1-(2-cyanoethyl))-3-methylimidazolium and/or an allylmethylimidazolium (e.g., N,N-allylmethylimidazolium); alternatively, the ionic liquid cation can comprise (1-(2-cyanoethyl))-3-methylimidazolium; alternatively, the ionic liquid cation can comprise an allylmethylimidazolium; or alternatively, the ionic liquid cation can comprise N,N-allylmethylimidazolium. Additionally or alternatively, the ionic liquid anion can comprise bis(trifluoromethanesulfonyl)imide.

The ionic liquid cations and anions are not limited solely to the selections disclosed hereinabove. Other suitable ionic liquid cations and anions are provided in U.S. Pat. Nos. 10,227,274, 9,732,016, and 9,238,193.

The metal-containing ionic liquid composition, in addition to the ionic liquid, contains a metal cation. Suitable metal cations can include, but are not limited to, a copper cation, a silver cation, a gold cation, a platinum cation, a palladium cation, an iridium cation, a rhenium cation, an alkali earth metal cation, and the like, as well as combinations thereof. In a particular aspect, the metal cation can comprise a copper cation, or a silver cation, or both copper and silver cations. For instance, the metal cation can comprise Cu(I), Ag(I), or both.

In certain aspects, the metal cation can be provided and present within the metal-containing ionic liquid composition as a metal salt comprising the metal cation and an anion. On loading the metal cation into the ionic liquid, the ionic liquid cation may exchange with the metal cation, thereby forming a metal complex between the ionic liquid anion and the metal cation. Advantageously, the metal cation can be provided as a salt or complex of the metal cation and an anion identical to the ionic liquid anion. For instance, Cu(I) bis(trifluoromethanesulfonyl)imide is an illustrative example of a metal salt that can be used to form the metal-containing ionic liquid composition. Other suitable ionic liquids that contain metal cations are disclosed in U.S. Pat. Nos. 10,227,274, 9,732,016, and 9,238,193.

The amount of the metal cation present in the metal-containing ionic liquid composition (or in the purified metal-containing ionic liquid composition) is not particularly limited, but nevertheless typically ranges from 0.5 M to 3 M. Other suitable ranges for the metal cation concentration include from 0.5 M to 2 M, from 1 M to 3 M, from 1 M to 2 M, from 1.5 to 2.5 M, or from 1.5 to 2 M, and the like.

The metal-containing ionic liquid composition also contains a solvent to reduce the viscosity of the composition, and the solvent is present in the composition at a solvent: metal cation molar ratio of at least 0.25:1. For instance, in one aspect, the molar ratio of solvent: metal cation is from 0.25:1 to 5:1, while in another aspect, the molar ratio is from 0.25:1 to 3:1, and in another aspect, the molar ratio is from 0.25:1 to 1:1, and in another aspect, the molar ratio is from 0.5:1 to 5:1, and in another aspect, the molar ratio is from 0.5:1 to 3:1, and in yet another aspect, the molar ratio is from 0.5:1 to 1:1, and in still another aspect, the molar ratio is from 1:1 to 2:1.

The solvent is a minor component of the metal-containing ionic liquid composition, often ranging from as little as 0.1 wt. % up to as much as 10 wt. %. More often, the solvent content of the metal-containing ionic liquid composition falls within a range from 0.1 wt. % to 5 wt. %, from 0.1 wt. % to 1 wt. %, from 0.5 wt. % to 10 wt. %, from 0.5 wt. % to 5 wt. %, from 1 wt. % to 10 wt. %, or from 1 wt. % to 5 wt. %.

The solvent serves to reduce the viscosity of the composition that often accompanies an increase in metal cation concentration in the composition. Often, a relatively small amount of the solvent can lead to significant reduction in viscosity, such as viscosity reductions over up to 50%, or more, with less than 10 wt. % of the solvent.

While not limited thereto, suitable solvents can include acetonitrile, dibenzyl ether, 3,4-dichlorotoluene, sulfolane, N-methylpyrrolidinone, caprolactone, propylene carbonate, benzylbutylphthalate. Combinations of two or more solvents can be utilized, if desired. In an aspect, the solvent in the metal-containing ionic liquid composition can comprise acetonitrile; alternatively, dibenzyl ether; alternatively, 3,4-dichlorotoluene; alternatively, sulfolane; alternatively, N-methylpyrrolidinone; alternatively, caprolactone; alternatively, propylene carbonate; or alternatively, benzylbutylphthalate.

Generally, due to presence of the solvent, the metal-containing ionic liquid composition can have a viscosity at 25° C. of less than or equal to 1000 cP. More often, the viscosity of the metal-containing ionic liquid composition at 25° C. is less than or equal to 500 cP or less than or equal to 200 cP, and representative ranges for the viscosity include from 100 to 500 cP and from 150 to 300 cP. Significantly, without the solvent, the metal-containing ionic liquid composition can have a viscosity of 500 to 1000 cP, and even higher viscosities are often encountered.

### Solvent Removal Processes

The processes disclosed herein generally are utilized to purify metal-containing ionic liquid compositions by removing some or all of the solvent component of the composition. In general, a suitable process can comprise contacting a metal-containing ionic liquid composition-which comprises (i) an ionic liquid comprising an ionic liquid cation and an ionic liquid anion, (ii) a metal cation, and (iii) a solvent at a molar ratio of solvent: metal cation of at least 0.25:1-with a gaseous unsaturated compound at a pressure of at least 50 psig to remove at least a portion of the solvent to form the purified metal-containing ionic liquid composition.

Referring first to the gaseous unsaturated compound, this terminology is meant to indicate that the gaseous unsaturated compound is a gas at standard temperature and pressure (25° C. and 1 atm). Suitable compounds are unsaturated. For instance, nitrogen (N₂) is ineffective at displacing the solvent compound of the metal-containing ionic liquid composition. The size of the gaseous unsaturated compound is not particularly limited, although smaller unsaturated compounds (e.g., lower carbon numbers) ordinarily are more volatile. Generally, the gaseous unsaturated compound is a C₁ to C₅ compound, such as C₁ to C₄ compound or a C₂ to C₃ compound, although not limited thereto.

Representative and non-limiting examples of suitable gaseous unsaturated compounds include ethylene, propylene, 1-butene, isobutylene, 4-methyl-1-pentene, acetylene, propyne, carbon dioxide, 1,1-difluoroethene, tetrafluoroethene, and the like. Combinations of two or more gaseous unsaturated compounds can be utilized, if desired. In an aspect, the gaseous unsaturated compound can comprise (or consist essentially of, or consist of) ethylene; alternatively, propylene; alternatively, 1-butene; alternatively, isobutylene; alternatively, 4-methyl-1-pentene; alternatively, acetylene; alternatively, propyne; alternatively, carbon dioxide; alternatively, 1,1-difluoroethene; or alternatively, tetrafluoroethene. Other fluorinated (or halogenated) ethenes in addition to 1,1-difluoroethene and tetrafluoroethene can be suitable gaseous unsaturated compounds for use in the processes disclosed herein.

Often, it is desirable to utilize a single gaseous unsaturated compound or a large majority of a single gaseous unsaturated compound. In such circumstances, the metal-containing ionic liquid composition can be contacted with a gaseous unsaturated compound comprising at least 85 vol % of a single gaseous unsaturated compound (e.g., ethylene), and more often, comprising at least 90 vol %, at least 95 vol %, at least 97 vol %, at least 98 vol %, at least 99 vol %, or at least 99.5 vol % of the single gaseous unsaturated compound (e.g., ethylene).

Any suitable conditions for contacting the metal-containing ionic liquid composition with the gaseous unsaturated compound can be utilized, so long as the conditions are appropriate for the gaseous unsaturated compound to displace at least a portion of the solvent present in the metal-containing ionic liquid composition. In some aspects, the gaseous unsaturated compound can be contacted with the metal-containing ionic liquid composition at a temperature in a range from 10° C. to 150° C., from 10° C. to 100° C., or from 10° C. to 70° C. In other aspects, the gaseous unsaturated compound can be contacted with the metal-containing ionic liquid composition at a temperature in a range from 20° C. to 150° C., from 20° C. to 100° C., or from 20° C. to 80° C. These temperature ranges also are meant to encompass circumstances where the gaseous unsaturated compound and the metal-containing ionic liquid composition are contacted at a series of different temperatures, instead of at a single fixed temperature, wherein at least one temperature falls within the respective ranges.

Likewise, the pressure at which the metal-containing ionic liquid composition and the gaseous unsaturated compound are contacted is not limited to a particular range. Generally, however, the pressure is at least 50 psig (and this total pressure is substantially the same as the partial pressure of the gaseous unsaturated compound). In some aspects, the gaseous unsaturated compound can be contacted with the metal-containing ionic liquid composition at a pressure in a range from 50 psig to 500 psig, from 50 psig to 250 psig, or from 75 psig to 250 psig (or at a partial pressure of the gaseous unsaturated compound in a range from 50 psig to 500 psig, from 50 psig to 250 psig, or from 75 psig to 250 psig). In other aspects, the gaseous unsaturated compound can be contacted with the metal-containing ionic liquid composition at a pressure in a range from 100 psig to 500 psig or from 100 to 250 psig (or at a partial pressure of the gaseous unsaturated compound in a range from 100 psig to 500 psig or from 100 to 250 psig).

As would be readily recognized, in addition to the temperature and the pressure under which the metal-containing ionic liquid composition and the gaseous unsaturated compound are contacted, the amount of time or duration of the contacting step can greatly impact the amount of solvent that will be displaced. While the duration of the contacting step can vary significantly, the metal-containing ionic liquid composition and the gaseous unsaturated compound can be contacted for a time period as brief as 30 sec to 1 min and for as long as 24 hr, or more. More often, the contacting step can occur and the purified metal-containing ionic liquid composition can be formed in a time period that ranges from 1 min to 18 hr, such as from 5 min to 12 hr, from 15 min to 8 hr, or from 30 min to 3 hr, and the like. To improve the contact between the gaseous unsaturated compound and the solvent in the composition, these components can be agitated and/or the gaseous unsaturated can flowed or bubbled through the (liquid phase) metal-containing ionic liquid composition, which contains the solvent.

After contacting the metal-containing ionic liquid composition with the gaseous unsaturated compound at a pressure of at least 50 psig and other appropriate conditions for removing at least a portion of the solvent, the disclosed processes can further comprise a step of reducing the pressure to vent/release the portion of the solvent removed and the (excess) gaseous unsaturated compound. Similar to the contacting step, any suitable conditions can be used, but these generally include a reduction in pressure, such as to ambient pressure or sub-ambient pressure. Absolute pressure ranges of from 5 psia to 50 psia or from 10 to 25 psia can be conveniently used.

As described above, prior to contacting with the gaseous unsaturated compound, the metal-containing ionic liquid composition can contain the solvent at molar ratios of solvent: metal cation from 0.25:1 to 5:1, such as from 0.25:1 to 3:1, from 0.25:1 to 1:1, from 0.5:1 to 5:1, from 0.5:1 to 3:1, from 0.5:1 to 1:1, or from 1:1 to 2:1. After contacting the gaseous unsaturated compound with the metal-containing ionic liquid composition, the resultant purified metal-containing ionic liquid composition can have a much lower amount of solvent, and generally this is reflected in a molar ratio of solvent: metal cation in the purified metal-containing ionic liquid composition of less than or equal to 0.1:1. In one aspect, the molar ratio of solvent: metal cation in the purified metal-containing ionic liquid composition is less than or equal to 0.08:1, while in another aspect, the molar ratio is less than or equal to 0.05:1, and in yet another aspect, the molar ratio is less than or equal to 0.03:1, and in still another aspect, the molar ratio is less than or equal to 0.02:1.

Generally, at least 50 wt. % of the solvent is removed from the metal-containing ionic liquid composition to form the purified metal-containing ionic liquid composition. More often, at least 80 wt. % or at least 90 wt. % of the solvent is removed from the metal-containing ionic liquid composition to form the purified metal-containing ionic liquid composition. In some aspects, at least 95 wt. % or at least 98 wt. % of the solvent is removed from the metal-containing ionic liquid composition to form the purified metal-containing ionic liquid composition, while in other aspects, at least 99 wt. % or at least 99.5 wt. % of the solvent is removed from the metal-containing ionic liquid composition to form the purified metal-containing ionic liquid composition.

Multiple contacting steps (followed by a respective vent/release step) can be applied to further increase the amount of solvent removed, as compared to a single contacting step. For instance, the process can further comprise a second cycle of contacting the metal-containing ionic liquid composition with the same or a different gaseous unsaturated compound at a pressure of at least 50 psig to remove at least a second portion of the solvent to form the purified metal-containing ionic liquid composition. Likewise, a third cycle, a fourth cycle, and so forth, can be utilized if desired.

The process can be conducted in any suitable vessel or apparatus, including combinations of two or more different vessels. In a non-limiting aspect, the metal-containing ionic liquid composition can be contacted with the gaseous unsaturated compound (at a pressure of at least 50 psig) in a pressure vessel.

### EXAMPLES

The disclosure is further illustrated by the following example, which is not to be construed in any way as imposing limitations to the scope of this disclosure. Various other aspects, modifications, and equivalents thereof, which after reading the description herein, can suggest themselves to one of ordinary skill in the art without departing from the spirit of the present disclosure or the scope of the appended claims.

### Constructive Example

A representative metal-containing ionic liquid composition contains an ionic liquid comprising an allylmethylimidazolium cation (e.g., N,N-allylmethylimidazolium) and a bis(trifluoromethanesulfonyl)imide anion. The metal anion is copper, and the metal salt precursor is copper(I) bis(trifluoromethanesulfonyl)imide. The metal cation (copper) concentration is 1.5-2.0 M in the composition.

The solvent in the metal-containing ionic liquid composition is acetonitrile. The molar ratio of solvent: metal cation is approximately 0.5:1. The amount of the solvent in the metal-containing ionic liquid composition is approximately 0.5 wt. %. The viscosity of the metal-containing ionic liquid composition at 25° C. is in the 150 to 300 cP range. Without the solvent, the viscosity of the metal-containing ionic liquid at 25° C. may be 1000 cP or more.

To remove the solvent from the metal-containing ionic liquid composition, the composition is placed in a pressure vessel (e.g., an autoclave reactor) at 25° C. and a gaseous unsaturated compound such as ethylene is flowed through the liquid and fed into the autoclave reactor until the ethylene pressure is 100 psig. Contact time in the autoclave reactor is 2 hr. Displaced solvent and excess ethylene is vented from the autoclave reactor. At least 90 wt. % of the solvent is removed from the metal-containing ionic liquid composition, thereby forming a purified metal-containing ionic liquid composition with a molar ratio of solvent: metal cation (acetonitrile: copper) in the purified metal-containing ionic liquid composition of less than 0.05:1.

The disclosure is described above with reference to numerous aspects and specific examples. Many variations will suggest themselves to those skilled in the art in light of the above detailed description. All such obvious variations are within the full intended scope of the appended claims. Other aspects of the disclosure can include, but are not limited to, the following (aspects are described as “comprising” but, alternatively, can “consist essentially of” or “consist of”):

Aspect 1. A process comprising contacting a metal-containing ionic liquid composition comprising (i) an ionic liquid comprising an ionic liquid cation and an ionic liquid anion, (ii) a metal cation, and (iii) a solvent at a molar ratio of solvent: metal cation of at least 0.25:1, with a gaseous unsaturated compound at a pressure of at least 50 psig to remove at least a portion of the solvent to form a purified metal-containing ionic liquid composition.

Aspect 2. The process defined in aspect 1, wherein the ionic liquid cation comprises an allylmethylimidazolium, a butylethylimidazolium, a butylmethylimidazolium, a butyldimethylimidazolium, a decaethylimidazolium, decamethylimidazolium, a diethylimidazolium, a dimethylimidazolium, an ethyl-2,4-dimethylimidazolium, an ethyldimethylimidazolium, an ethylimidazolium, an ethylmethylimidazolium, an ethylpropylimidazolium, an ethoxyethylmethylimidazolium, an ethoxydimethylimidazolium, a hexadecylmethylimidazolium, a heptylmethylimidazolium, a hexylethylimidazolium, a hexylmethylimidazolium, a hexyldimethylimidazolium, a methoxyethylmethylimidazolium, a methoxypropylmethylimidazolium, a methylimidazolium, a dimethylimidazolium, a methylnonylimidazolium, a methylnonylimidazolium, an octadecylmethylimidazolium, a hydroxylethylmethylimidazolium, a hydroxyloctylmethylimidazolium, a hydroxylpropylmethylimidazolium, an octylmethylimidazolium, an octyldimethylimidazolium, a phenylethylmethylimidazolium, a phenylmethylimidazolium, a phenyldimethylimidazolium, a pentylmethylimidazolium, a propylmethylimidazolium, 1-butyl-2-methylpyridinium, 1-butyl-3-methylpyridinium, a butylmethylpyridinium, 1-butyl-4-dimethylacetylpyridinium, 1-butyl-4-methylpyridinium, 1-ethyl-2-methylpyridinium, 1-ethyl-3-methylpyridinium, 1-ethyl-4-dimethylacetylpyridinium, 1-ethyl-4-methylpyridinium, 1-hexyl-4-dimethylacetylpyridinium, 1-hexyl-4-methylpyridinium, 1-octyl-3-methylpyridinium, 1-octyl-4-methylpyridinium, 1-propyl-3-methylpyridinium, 1-propyl-4-methylpyridinium, a butylpyridinium, an ethylpyridinium, a heptylpyridinium, a hexylpyridinium, a hydroxypropylpyridinium, an octylpyridinium, a pentylpyridinium, a propylpyridinium, a butylmethylpyrrolidinium, a butylpyrrolidinium, a hexylmethylpyrrolidinium, a hexylpyrrolidinium, an octylmethylpyrrolidinium, an octylpyrrolidinium, a propylmethylpyrrolidinium, a butylammonium, tributylammonium, tetrabutylammonium, butylethyldimethylammonium, butyltrimethylammonium, N,N,N-trimethylethanolammonium, ethylammonium, diethylammonium, tetraethylammonium, tetraheptylammonium, tetrahexylammonium, methylammonium, dimethylammonium, tetramethylammonium, ammonium, butyldimethylethanolammonium, dimethylethanolammonium, ethanolammonium, ethyldimethylethanolammonium, a tetrapentylammonium, tetrapropylammonium, a tetrabutylphosphonium, a tributyloctylphosphonium, or any combination thereof.

Aspect 3. The process defined in aspect 1 or 2, wherein the ionic liquid anion comprises bis(trifluoromethanesulfonyl)imide, bis(fluorosulfonyl)imide, hexafluorophosphate, trifluoromethanesulfonate, dicyanamide, tetrafluoroborate, thiocyanate, nitrate, sulfonate, methylsulfate, methanesulfonate, trifluoroacetate, acetate, or any combination thereof.

Aspect 4. The process defined in any one of aspects 1-3, wherein the ionic liquid cation comprises an allylmethylimidazolium, an ethylmethylimidazolium, (1-(2-cyanoethyl))-3-methylimidazolium, a propenylmethylimidazolium, a propargylmethylimidazolium, a butylmethylimidazolium, a butylmethylpyridinium, or any combination thereof, and the ionic liquid anion comprises bis(trifluoromethanesulfonyl)imide, hexafluorophosphate, trifluoromethanesulfonate, dicyanamide, tetrafluoroborate, thiocyanate, nitrate, sulfonate, methylsulfate, or any combination thereof.

Aspect 5. The process defined in any one of aspects 1-4, wherein the ionic liquid cation comprises (1-(2-cyanoethyl))-3-methylimidazolium and/or N,N-allylmethylimidazolium, and the ionic liquid anion comprises bis(trifluoromethanesulfonyl)imide.

Aspect 6. The process defined in any one of aspects 1-5, wherein the metal cation comprises a copper cation and/or a silver cation.

Aspect 7. The process defined in any one of aspects 1-6, wherein the metal cation is provided as a metal salt.

Aspect 8. The process defined in aspect 7, wherein the metal salt is copper (I) bis(trifluoromethanesulfonyl)imide.

Aspect 9. The process defined in any one of aspects 1-8, wherein the metal-containing ionic liquid composition (or the purified metal-containing ionic liquid composition) has a metal cation concentration in any range disclosed herein, e.g., from 0.5 M to 3 M, from 0.5 M to 2 M, from 1 M to 3 M, from 1 M to 2 M, from 1.5 to 2.5 M, or from 1.5 to 2 M.

Aspect 10. The process defined in any one of aspects 1-9, wherein the molar ratio of solvent: metal cation is in any range disclosed herein, e.g., from 0.25:1 to 5:1, from 0.25:1 to 3:1, from 0.25:1 to 1:1, from 0.5:1 to 5:1, from 0.5:1 to 3:1, from 0.5:1 to 1:1, or from 1:1 to 2:1.

Aspect 11. The process defined in any one of aspects 1-10, wherein an amount of the solvent in the metal-containing ionic liquid composition is in any range disclosed herein, e.g., from 0.1 wt. % to 10 wt. %, from 0.1 wt. % to 5 wt. %, from 0.1 wt. % to 1 wt. %, from 0.5 wt. % to 10 wt. %, from 0.5 wt. % to 5 wt. %, from 1 wt. % to 10 wt. %, or from 1 wt. % to 5 wt. %.

Aspect 12. The process defined in any one of aspects 1-11, wherein the solvent comprises acetonitrile, dibenzyl ether, 3,4-dichlorotoluene, sulfolane, N-methylpyrrolidinone, caprolactone, propylene carbonate, benzylbutylphthalate, or any combination thereof.

Aspect 13. The process defined in any one of aspects 1-12, wherein the solvent comprises acetonitrile, dibenzyl ether, or a combination thereof.

Aspect 14. The process defined in any one of aspects 1-13, wherein the metal-containing ionic liquid composition has a viscosity at 25° C. in any range disclosed herein, e.g., less than or equal to 1000 cP, less than or equal to 500 cP, less than or equal to 200 cP, from 100 to 500 cP, or from 150 to 300 cP.

Aspect 15. The process defined in any one of aspects 1-14, wherein the gaseous unsaturated compound is a gas at standard temperature and pressure (25° C. and 1 atm).

Aspect 16. The process defined in any one of aspects 1-15, wherein the gaseous unsaturated compound is a C₁ to C₅ compound, a C₁ to C₄ compound, or a C₂ to C₃ compound.

Aspect 17. The process defined in any one of aspects 1-16, wherein the gaseous unsaturated compound comprises ethylene, propylene, 1-butene, isobutylene, 4-methyl-1-pentene, acetylene, propyne, carbon dioxide, 1,1-difluoroethene, tetrafluoroethene, or any combination thereof.

Aspect 18. The process defined in any one of aspects 1-17, wherein the pressure is in any range disclosed herein, e.g., from 50 psig to 500 psig, from 50 psig to 250 psig, from 75 psig to 250 psig, from 100 psig to 500 psig, or from 100 to 250 psig.

Aspect 19. The process defined in any one of aspects 1-18, wherein the metal-containing ionic liquid composition and the gaseous unsaturated compound are contacted at a temperature in any range disclosed herein, e.g., from 10° C. to 150° C., from 10° C. to 100° C., from 10° C. to 70° C., from 20° C. to 150° C., from 20° C. to 100° C., or from 20° C. to 80° C.

Aspect 20. The process defined in any one of aspects 1-19, wherein a molar ratio of solvent: metal cation in the purified metal-containing ionic liquid composition is in any range disclosed herein, e.g., less than or equal to 0.1:1, less than or equal to 0.08:1, less than or equal to 0.05:1, less than or equal to 0.03:1, or less than or equal to 0.02:1.

Aspect 21. The process defined in any of aspects 1-20, wherein any percentage amount disclosed herein of the solvent is removed from the metal-containing ionic liquid composition to form the purified metal-containing ionic liquid composition, e.g., at least 50 wt. %, at least 80 wt. %, at least 90 wt. %, at least 95 wt. %, at least 98 wt. %, at least 99 wt. %, or at least 99.5 wt. %.

Aspect 22. The process defined in any of aspects 1-21, further comprising a step of reducing the pressure to vent/release the portion of the solvent removed and the gaseous unsaturated compound.

Aspect 23. The process defined in any of aspects 1-22, further comprising a second cycle of contacting the metal-containing ionic liquid composition with the same or a different gaseous unsaturated compound at a pressure of at least 50 psig to remove at least a second portion of the solvent to form the purified metal-containing ionic liquid composition.

Aspect 24. The process defined in any of aspects 1-23, wherein the process in conducted in a pressure vessel.

What is claimed is:

1. A process comprising:
contacting a metal-containing ionic liquid composition comprising:
(i) an ionic liquid comprising an ionic liquid cation and an ionic liquid anion;
(ii) a metal cation; and
(iii) a solvent at a molar ratio of solvent: metal cation of at least 0.25:1;
with a gaseous unsaturated compound at a pressure of at least 50 psig to remove at least a portion of the solvent to form a purified metal-containing ionic liquid composition.

2. The process of claim 1, wherein:
the ionic liquid cation comprises an allylmethylimidazolium, a butylethylimidazolium, a butylmethylimidazolium, a butyldimethylimidazolium, a decaethylimidazolium, a decamethylimidazolium, a diethylimidazolium, a dimethylimidazolium, an ethyl-2,4-dimethylimidazolium, an ethyldimethylimidazolium, an ethylimidazolium, an ethylmethylimidazolium, an ethylpropylimidazolium, an ethoxyethylmethylimidazolium, an ethoxydimethylimidazolium, a hexadecylmethylimidazolium, a heptylmethylimidazolium, a hexylethylimidazolium, a hexylmethylimidazolium, a hexyldimethylimidazolium, a methoxyethylmethylimidazolium, a methoxypropylmethylimidazolium, a methylimidazolium, a dimethylimidazolium, a methylnonylimidazolium, a methylnonylimidazolium, an octadecylmethylimidazolium, a hydroxylethylmethylimidazolium, a hydroxyloctylmethylimidazolium, a hydroxylpropylmethylimidazolium, an octylmethylimidazolium, an octyldimethylimidazolium, a phenylethylmethylimidazolium, a phenylmethylimidazolium, a phenyldimethylimidazolium, a pentylmethylimidazolium, a propylmethylimidazolium, 1-butyl-2-methylpyridinium, 1-butyl-3-methylpyridinium, a butylmethylpyridinium, 1-butyl-4-dimethylacetylpyridinium, 1-butyl-4-methylpyridinium, 1-ethyl-2-methylpyridinium, 1-ethyl-3-methylpyridinium, 1-ethyl-4-dimethylacetylpyridinium, 1-ethyl-4-methylpyridinium, 1-hexyl-4-dimethylacetylpyridinium, 1-hexyl-4-methylpyridinium, 1-octyl-3-methylpyridinium, 1-octyl-4-methylpyridinium, 1-propyl-3-methylpyridinium, 1-propyl-4-methylpyridinium, a butylpyridinium, an ethylpyridinium, a heptylpyridinium, a hexylpyridinium, a hydroxypropylpyridinium, an octylpyridinium, a pentylpyridinium, a propylpyridinium, a butylmethylpyrrolidinium, a butylpyrrolidinium, a hexylmethylpyrrolidinium, a hexylpyrrolidinium, an octylmethylpyrrolidinium, an octylpyrrolidinium, a propylmethylpyrrolidinium, a butylammonium, tributylammonium, tetrabutylammonium, butylethyldimethylammonium, butyltrimethylammonium, N,N,N-trimethylethanolammonium, ethylammonium, diethylammonium, tetraethylammonium, tetraheptylammonium, tetrahexylammonium, methylammonium, dimethylammonium, tetramethylammonium, ammonium, butyldimethylethanolammonium, dimethylethanolammonium, ethanolammonium, ethyldimethylethanolammonium, a tetrapentylammonium, tetrapropylammonium, a tetrabutylphosphonium, a tributyloctylphosphonium, or any combination thereof; and
the ionic liquid anion comprises bis(trifluoromethanesulfonyl)imide, bis(fluorosulfonyl)imide, hexafluorophosphate, trifluoromethanesulfonate, dicyanamide, tetrafluoroborate, thiocyanate, nitrate, sulfonate, methylsulfate, methanesulfonate, trifluoroacetate, acetate, or any combination thereof.

3. The process of claim 1, wherein:
the ionic liquid cation comprises an allylmethylimidazolium, an ethylmethylimidazolium, (1-(2-cyanoethyl))-3-methylimidazolium, a propenylmethylimidazolium, propargylmethylimidazolium, a butylmethylimidazolium, a butylmethylpyridinium, or any combination thereof; and
the ionic liquid anion comprises bis(trifluoromethanesulfonyl)imide, hexafluorophosphate, trifluoromethanesulfonate, dicyanamide, tetrafluoroborate, thiocyanate, nitrate, sulfonate, methylsulfate, or any combination thereof.

4. The process of claim 1, wherein:
the ionic liquid cation comprises (1-(2-cyanoethyl))-3-methylimidazolium and/or N,N-allylmethylimidazolium; and
the ionic liquid anion comprises bis(trifluoromethanesulfonyl)imide.

5. The process of claim 1, wherein the metal cation comprises a copper cation and/or a silver cation.

6. The process of claim 1, wherein the metal cation is provided as a metal salt.

7. The process of claim 6, wherein the metal salt is copper(I) bis(trifluoromethanesulfonyl)imide.

8. The process of claim 1, wherein the metal-containing ionic liquid composition or the purified metal-containing ionic liquid composition has a metal cation concentration in a range from 0.5 M to 3 M.

9. The process of claim 1, wherein a molar ratio of solvent: metal cation is in a range from 0.25:1 to 5:1.

10. The process of claim 1, wherein an amount of the solvent in the metal-containing ionic liquid composition is in a range from 0.1 wt. % to 10 wt. %.

11. The process of claim 1, wherein the solvent comprises acetonitrile, dibenzyl ether, 3,4-dichlorotoluene, sulfolane, N-methylpyrrolidinone, caprolactone, propylene carbonate, benzylbutylphthalate, or any combination thereof.

12. The process of claim 1, wherein the metal-containing ionic liquid composition has a viscosity at 25° C. of less than or equal to 1000 cP.

13. The process of claim 1, wherein the gaseous unsaturated compound is a gas at standard temperature (25° C.) and pressure (1 atm).

14. The process of claim 1, wherein:
the gaseous unsaturated compound is a C₁ to C₅ compound; and/or the gaseous unsaturated compound comprises ethylene, propylene, 1-butene, isobutylene, 4-methyl-1-pentene, acetylene, propyne, carbon dioxide, 1,1-difluoroethene, tetrafluoroethene, or any combination thereof.

15. The process of claim 1, wherein:
the pressure is in a range from 50 psig to 500 psig; and/or
the metal-containing ionic liquid composition and the gaseous unsaturated compound are contacted at a temperature in a range from 10° C. to 150° C.

16. The process of claim 1, wherein a molar ratio of solvent: metal cation in the purified metal-containing ionic liquid composition is less than or equal to 0.1:1.

17. The process of claim 1, wherein at least 50 wt. % of the solvent is removed from the metal-containing ionic liquid composition to form the purified metal-containing ionic liquid composition.

18. The process of claim 1, further comprising a step of reducing the pressure to vent or release the portion of the solvent removed and the gaseous unsaturated compound.

19. The process of claim 1, further comprising a second cycle of contacting the metal-containing ionic liquid composition with the same or a different gaseous unsaturated compound at a pressure of at least 50 psig to remove at least a second portion of the solvent to form the purified metal-containing ionic liquid composition.

20. The process of claim 1, wherein the process in conducted in a pressure vessel.
